(19) United States
(12) Patent Application Publication
(10) Pub. No.: US 2010/0035925 A1
(43) Pub. Date: Feb. 11, 2010

(54) COMPOUNDS, PHARMACEUTICAL COMPOSITIONS AN DMETHODS FOR INHIBITING HIV INFECTIVITY

(75) Inventors: Michael Goldblatt, Gaithersburg, MD (US); Michael S. Kinch, Gaithersburg, MD (US); Limin Li, Potomac, MD (US)

Correspondence Address: Steven B. Kelber;Berenato, White & Stavish, 6550 Rock Spring Drive, Suite 240, Bethesda, MD, 20817, US

(73) Assignee: FUNCTIONAL GENETICS, INC, Gaithersburg, MD (US)

(21) Appl. No.: 12/539,741

(22) Filed: Aug. 12, 2009

Related U.S. Application Data
(63) Continuation-in-part of application No. 11/166,726, filed on Jun. 27, 2005.
(60) Provisional application No. 60/582,533, filed on Jun. 25, 2004.

(51) Int. Cl. A61K 31/4706; A61P 31/18

(52) U.S. Cl. 514/313

(57) ABSTRACT

Compounds that possess anti-infective activity are described. Methods of using these compounds for the treatment or prevention of infectious diseases such as acquired immunodeficiency syndrome (AIDS) are also described. The compounds inhibit HIV infectivity and do not exhibit significant cytotoxicity in HIV producing cells.

### CROSS REFERENCE TO RELATED CASES

This application claims the benefit of U.S. patent application Ser. No. 11/166,726 filed Jun. 27, 2005 (pending) and through that application, benefit of Provisional U.S. Application Ser. No. 60/582,533, filed Jun. 25, 2004. The disclosures of both these priority cases are incorporated by reference herein in its entirety.

### BACKGROUND

1. Technical Field

This application relates generally to pharmaceutical compositions and methods of treatment and, in particular, to compounds, pharmaceutical compositions and methods for inhibiting HIV infectivity.

2. Background of the Technology

HIV and other retroviruses begin their infectious life cycle by binding to cell surface proteins, an adhesion that promotes fusion of the viral and cell membranes and entry of the viral genome (its complete set of instructions for the manufacture of mature viral progeny) into the cell. Here, the RNA-based viral genome replicates and, after integrating into a host chromosome, directs the production of new viral RNA and proteins. These viral components then self-assemble and escape from the cell as mature viral progeny. From adhesion to escape, the infectious cycle requires the concerted activity of viral and cellular proteins. One such cellular protein is TSG101.

To propagate, fully assembled viruses must “bud” and pinch off membranous material from the cell surface. It has been shown that viruses are unable to carry out this final stage of maturation, and therefore recruit TSG101 and other cellular proteins for assistance. Normally, TSG101 directs proteins to their appropriate locations within the cell. In HIV-infected cells, however, this protein “traffic cop” becomes an unwitting though essential player in the viral life cycle, escorting viral particles to the cell membrane for eventual release.

Current strategies for the treatment of HIV and other viral diseases limit viral propagation by selectively inhibiting viral proteins. But the genes encoding these viral proteins (including reverse transcriptase and protease, for example) frequently mutate, rendering the viruses resistant to the effects of inhibitors. Thus, while this virus-centered pharmacological treatment may limit the absolute number of viruses in a patient (the viral load) the emergence of drug-resistant strains continues to undermine the therapeutic management of viral disease.

Accordingly, there still exists a need for improved pharmacological treatments for HIV and other infectious diseases.

### SUMMARY

According to a first embodiment, a pharmaceutical composition is provided which comprises: a compound represented by the formula (a), (b), (c), (d), (e), (f) or (g) below;

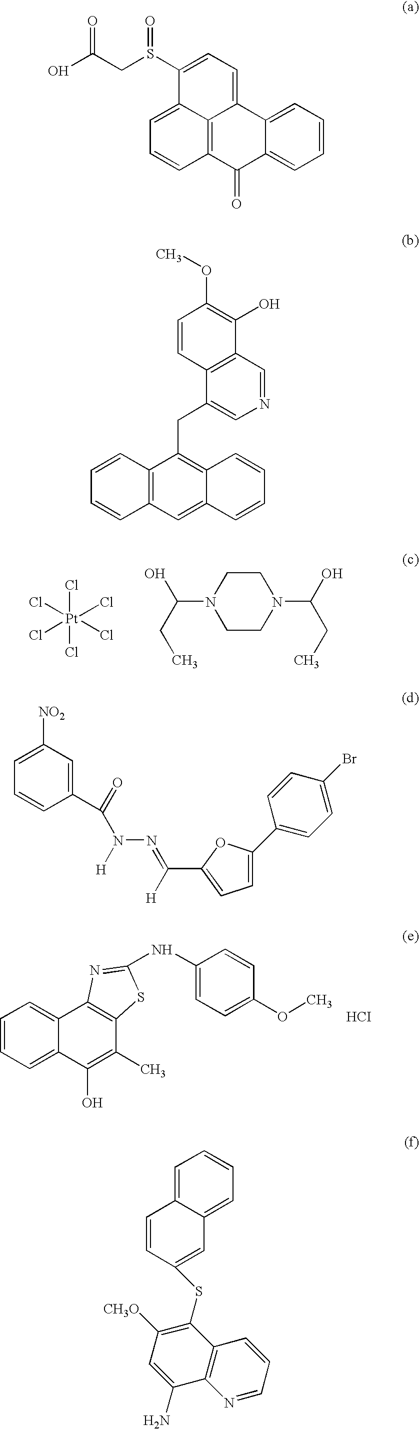

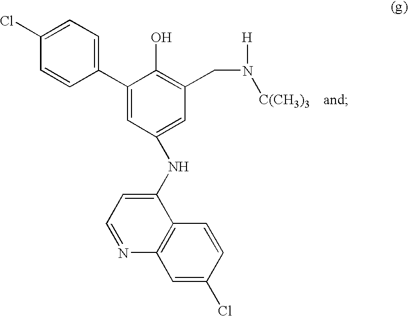

a pharmaceutically acceptable carrier or excipient.

Compounds related to these compounds have previously been investigated for their effectiveness in the treatment of malaria—see, e.g., Werbel et al., J. Med. Chem. 29, 924-939 (1986) although ultimately, these compounds were not adopted as pharmaceuticals. In general, their use to treat malaria parasites required dosage values ten times the effective dosage to treat viruses. (Effective dosage rates are from 0.1-to below 1.0 mg/kg of body weight for many viruses).

According to a second embodiment, a method for inhibiting HIV infectivity in a human is provided which comprises administering an effective amount of a compound as set forth above to a human.

According to a third embodiment, a method for preparing a composition is provided which comprises admixing a compound as set forth above with a pharmaceutically acceptable excipient or carrier.

According to a fourth embodiment, a method for inhibiting HIV infectivity is provided which comprises administering a therapeutically effective amount of a compound as set forth above to a patient in need thereof.

### BRIEF DESCRIPTION OF THE DRAWINGS

FIG. 1 is a graph showing percent inhibition of VP40/TSG101 binding by compounds 111, 1334 and 2958 wherein % inhibition is plotted as a function of compound concentration.

FIG. 2 is a graph showing percent inhibition of VP40/TSG101 binding by compound 2958 wherein % inhibition is plotted as a function of compound concentration.

FIG. 3 is a graph showing the inhibition of HIV infectivity by compounds 111, 1334 and 2958 wherein relative luciferase activity is plotted as a function of compound concentration.

FIG. 4 is a graph showing the cytotoxicity of compounds 111, 1334 and 2958 on transfected 293 cells wherein relative fluorescence units is plotted as a function of compound concentration.

FIG. 5 is a graph showing inhibition of HIV infectivity by compounds 111, 1334 and 2958 on TZM cells wherein relative luciferase activity is plotted versus the concentration of the compounds.

FIG. 6A is a graph showing the cytotoxicity of compounds 111, 1334 and 2958 on Jurkat-B2N cells wherein relative fluorescence units is plotted versus the concentration of the compounds.

FIG. 6B is a graph showing the cytotoxicity of compounds 111, 1334 and 2958 on TZM cells wherein relative fluorescence units is plotted versus the concentration of the compounds.

FIG. 7 is a graph showing inhibition of HIV activity for compounds R387401, R2, R10, and R11 wherein relative luciferase activity is plotted as a function of the concentration of each compound.

FIG. 8 is a graph illustrating cytotoxicity for compounds R387401, R2, R10, and R11 wherein percentage of cytotoxicity is plotted as a function of the concentration of each compound.

### DETAILED DESCRIPTION

Three lead small molecules were identified for their ability to inhibit TSG101 and Ebola VP40 interaction using a VP40/TSG101 binding assay. These compounds are set forth below:

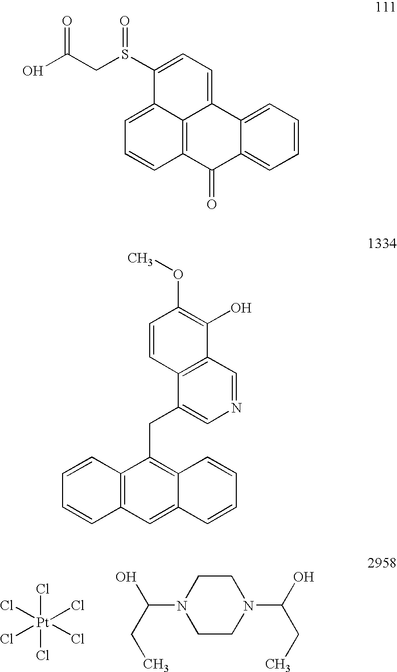

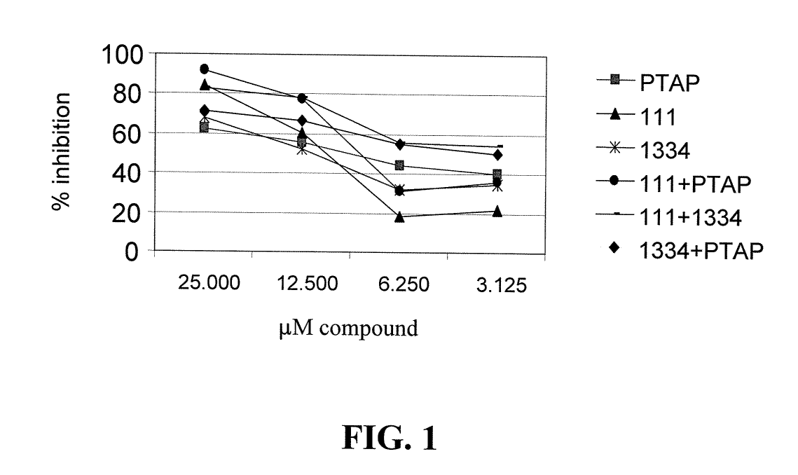

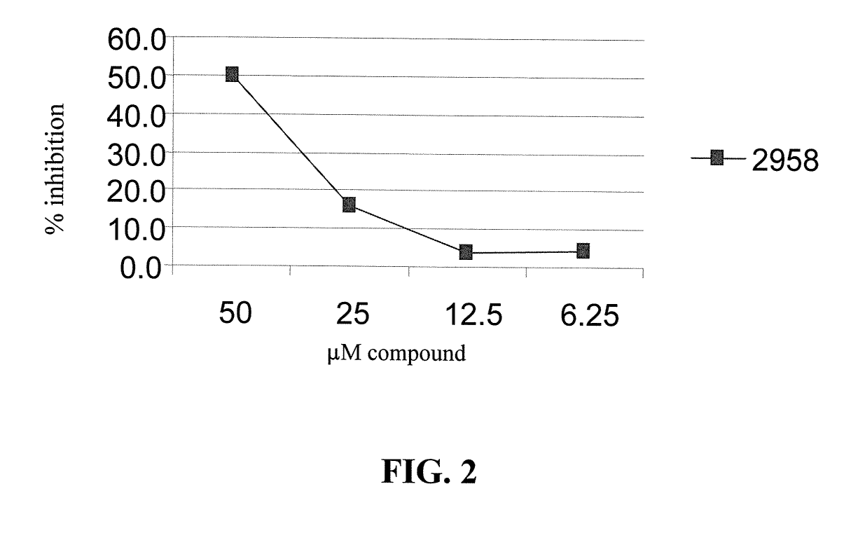

Compound III has been designated NSC-16211 by the Cancer Chemotherapy National Service Center (CCNSC). Compound 1334 has been designated NSC-131734 and compound 2958 has been designated NSC-295558 by the CCNSC. The results of the VP40/TSG101 binding assay are shown in FIGS. 1 and 2.

To determine if these three small molecules also inhibited HIV infectivity, these compounds were tested with two HIV drug resistant strains (pL10R and p1617-1, NIH AIDS Research & Reference Reagent Program). DNA plasmids pL10R and p1617-1 were transfected into HEK 293 cells by Fugen 6 (Roche) at 0.1 μg DNA per well in 96-well plate. Twenty-four (24) hours later, the cultures were washed with fresh medium, and three small molecules at different concentrations were added to each well. Following 48 hours incubation, 20 μl of cell culture supernatant from each well was collected and used to determine infectious HIV particles using standard MAGI assay (Wei et al., Antimicrob. Agents Chemother., 46: pp. 1896-1905, 2002). The inhibition of HIV infectivity was determined by assaying Luciferase activity in MAGI cells.

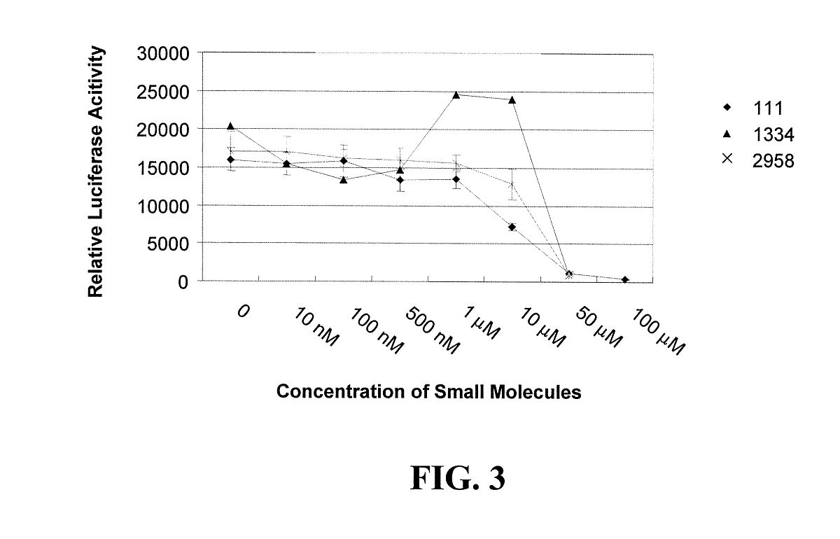

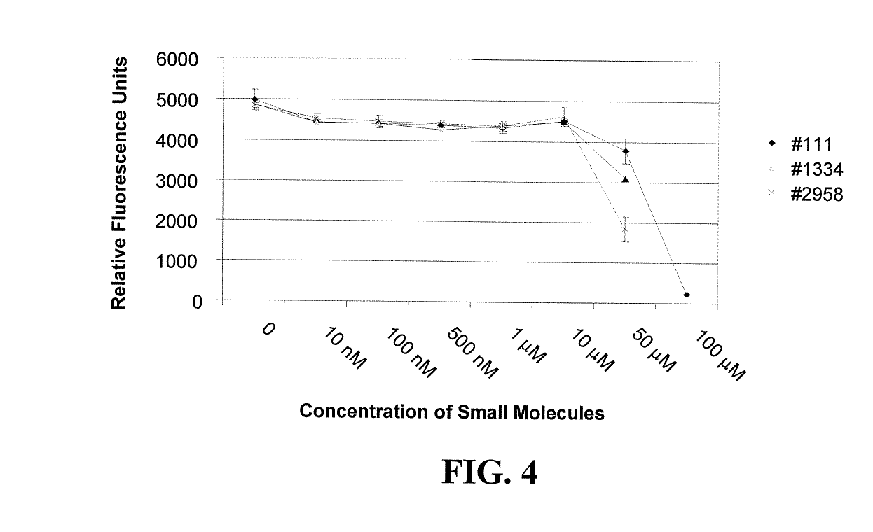

As shown in FIG. 3 and FIG. 4, small molecule 111 showed inhibitory activity at concentrations from 1 μM to 10 μM without significant cytotoxicity (each assay point represented the average of 6 replicate assays, and the variation of the assays was showed with standard deviation (error bars). A lesser inhibitory activity was also observed in 1334, but no significant inhibitory activity was detected with 2958 in these experiments.

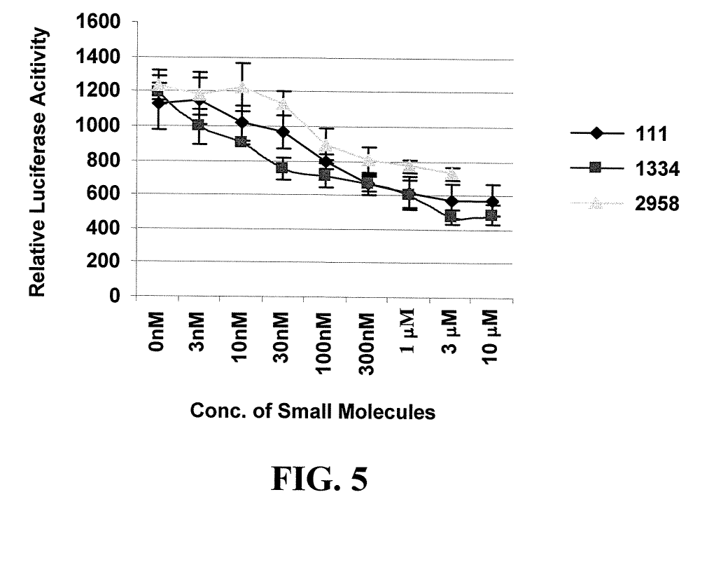

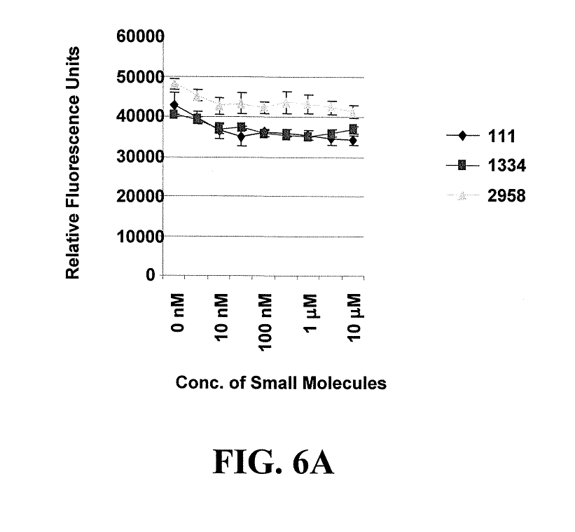

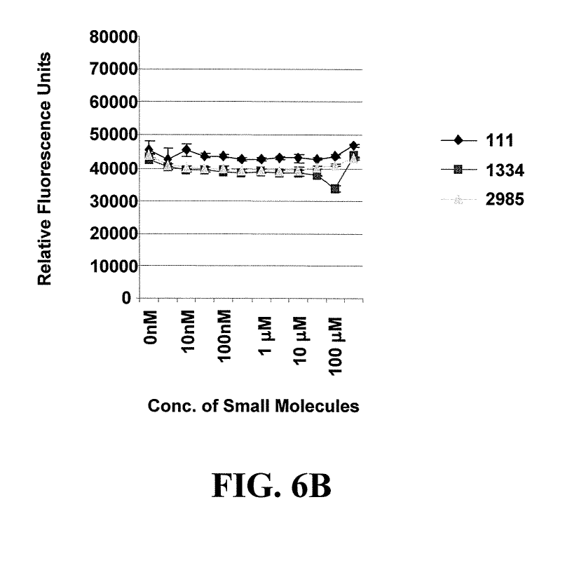

To further validate the these results, the three small molecule leads were tested against an HIV producing T lymphocyte line Jurkat cells. The results of this assay are shown in FIG. 5. All three molecule leads showed dosage dependent inhibition to HIV infectivity, with LD50 at 1 μM for 111 and 1334, and more than 3 μM for 2958. As shown in FIG. 6A and FIG. 6B, none of the small molecules produced significant cytotoxicity in HIV producing cells and MAGI up to 10 μM.

The ability of these small molecules, and their derivative families, to inhibit HIV infectivity in the absence of cytotoxicity offers a fundamental new, and promising, alternative to HIV treatment that may profoundly alter our approach to this and related pervasive diseases.

Other small molecules were also found to inhibit HIV infectivity. These small molecules include:

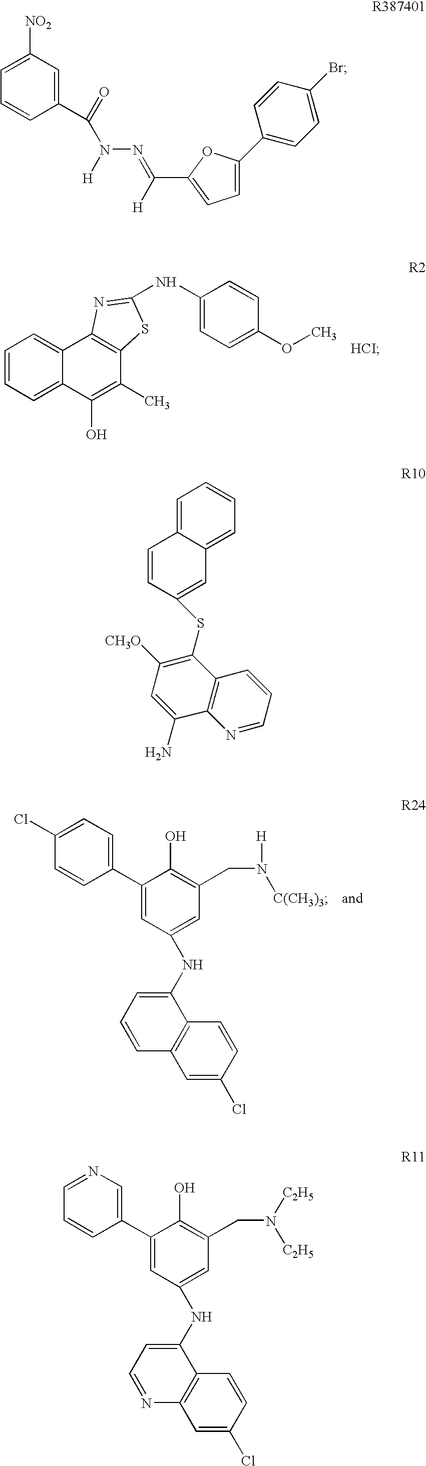

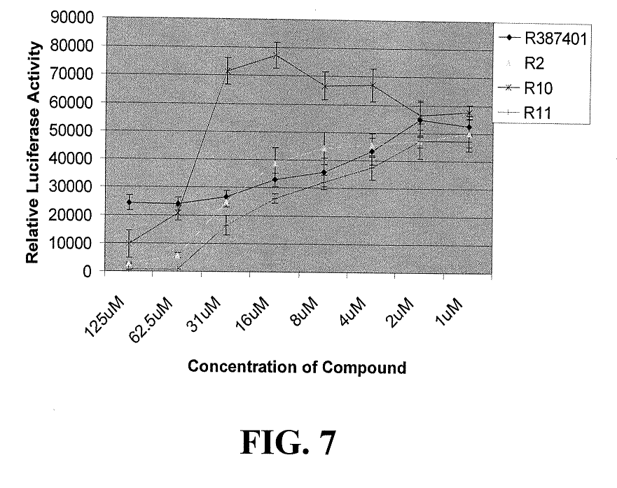

FIG. 7 is a graph showing inhibition of HIV activity for compounds R387401, R2, R10, and R11 wherein relative luciferase activity is plotted as a function of the concentration of each compound. As can be seen from FIG. 7, decreasing concentrations of the compounds generally result in higher relative luciferase activity.

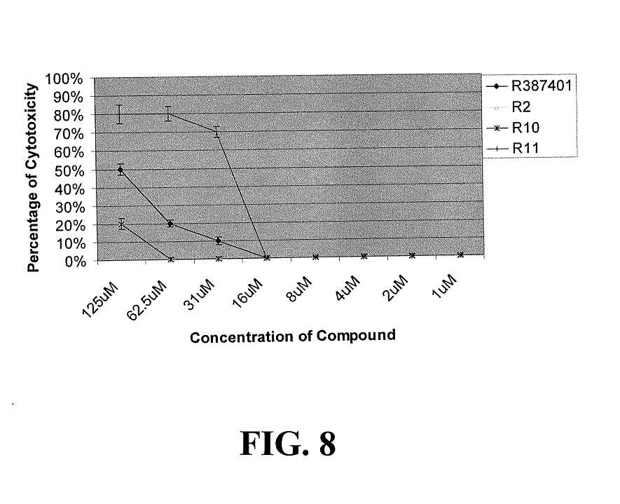

FIG. 8 is a graph illustrating cytotoxicity for compounds R387401, R2, R10, and R11 wherein percentage of cytotoxicity is plotted as a function of the concentration of each compound. As can be seen from FIG. 8, none of the compounds tested exhibited significant cytotoxicity at concentrations of about 10:M or less.

Various non-limiting exemplary embodiments are described below.

According to a first embodiment, a pharmaceutical composition is provided which comprises: a compound represented by the formula (a), (b), (c), (d), (e), (f) or (g) below;

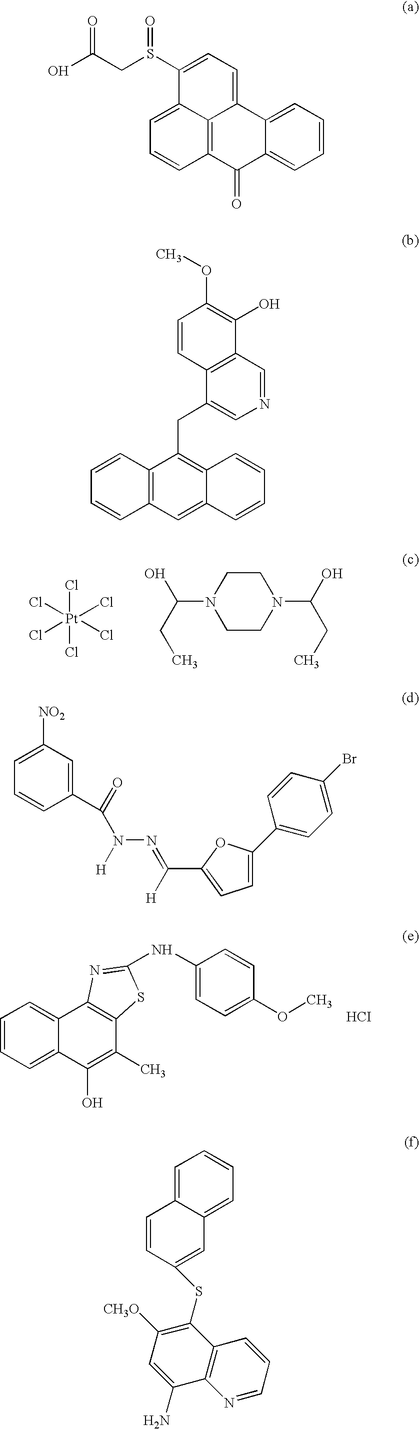

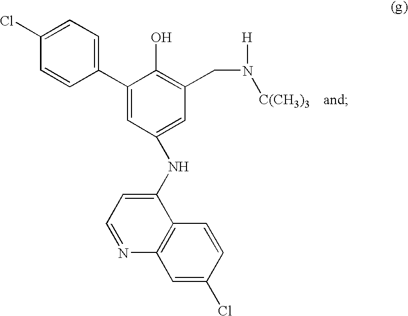

a pharmaceutically acceptable carrier or excipient.

The compounds may be modified from the structures set forth. The hetero atoms in the aryl ring of the compounds need not be present—at least one hetero atom may be replaced by a carbon atom in each ring without loss of activity. Each ring substituent may independently be H or an electron donating group, which may be selected from the group including chloro or other halogen, alkoxy (—OR), hydroxyl (—OH), aryloxy (—OAr), trialkylammonium (—NR₃⁺), alkylamido (—NHCOR, —NRCOR), arylamido (—NHCOAr, —NRCOAr, —NArCOAr), arylcarbamoyl (—NHCOOAr, —NRCOOAr), alkylcarbamoyl (—NHCOOR, —NRCOOR), cyano (—CN), nitro (—NO₂), ester (—COOR, —COOAr), or alkyl halo, wherein each R is an alkyl or branched alkyl of 1-6 carbon atoms. Each phenyl ring may also bear a substituent which is an alkyl of 1-4 carbon atoms, hydroxy, alkoxy or methylene. One phenyl ring may bear a di- or -tri akly amino, or alkyl di or tri amino, optionally substituted with a halogen moiety.

According to a second embodiment, a method for inhibiting HIV infectivity in a human is provided which comprises administering an effective amount of a compound as set forth above to a human. The human can be infected with HIV or exposed to the HIV virus.

According to a third embodiment, a method for preparing a composition is provided which comprises admixing a compound as set forth above with a pharmaceutically acceptable excipient or carrier.

According to a fourth embodiment, a method for inhibiting HIV infectivity is provided which comprises administering a therapeutically effective amount of a compound as set forth above to a patient in need thereof.

While the foregoing specification teaches the principles of the present invention, with examples provided for the purpose of illustration, it will be appreciated by one skilled in the art from reading this disclosure that various changes in form and detail can be made without departing from the true scope of the invention.

What is claimed is:

1. A method of inhibiting Human Immunodeficiency Virus (HIV) infectivity in a mammal, comprising administering to said mammal a dosage of a compound of the following formula in amounts effective to inhibit HIV infectivity in said mammal: 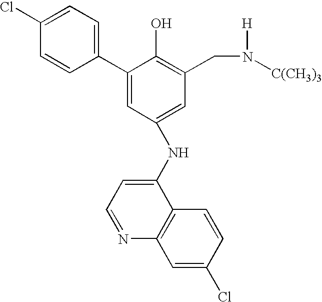
wherein each chlorine may independently be replaced with one electron donating group selected from the group including non-chloro halo, alkoxy (—OR), hydroxyl (—OH), aryloxy (—OAr), trialkylammonium (—NR₃⁺), alkylamido (—NHCOR, —NRCOR), arylamido (—NHCOAr, —NRCOAr, —NArCOAr), arylcarbamoyl (—NHCOOAr, —NRCOOAr), alkylcarbamoyl (—NHCOOR, —NRCOOR), cyano (—CN), nitro (—NO₂), ester (—COOR, —COOAr), or alkylhalo wherein each R is an alkyl or branched alkyl of 1-6 carbon atoms

2. The method of claim 1, wherein the compound is represented by the formula

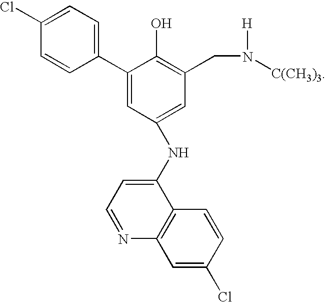

3. A method of inhibiting HIV infectivity in a mammal, comprising:
1) selecting a mammal in need of treatment for HIV infection, and
2) administering to said selected mammal an amount of the compound 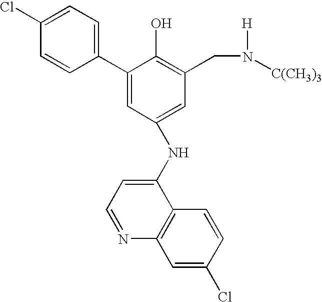
effective to inhibit HIV infectivity.
